(19) United States
(12) Patent Application Publication
(10) Pub. No.: US 2018/0098615 A1
(43) Pub. Date: Apr. 12, 2018

(54) COSMETIC COMPOSITIONS WITH SENSORIAL AND AESTHETIC BENEFITS AND HAVING ENHANCED STABILITY

(71) Applicant: L'OREAL, Paris (FR)

(72) Inventors: Kelly Marie George, Denville, NJ (US); Hy Si Bui, Piscataway, NJ (US); Aaron David Poole, Federal Way, WA (US); Mikeal Poletti, Paris (FR); Gerald Keith Brewer, Redmond, WA (US)

(21) Appl. No.: 15/224,057

(22) Filed: Oct. 11, 2016

(51) Int. Cl. A45D 40/26; A61K 8/02; A61Q 5/02; A61Q 19/10; A61Q 3/00; A45D 19/00; A46B 13/00; A46B 13/02; A46B 9/06; A46B 5/00; A46B 15/00; A47K 7/04

(52) U.S. Cl. CPC A45D 40/262; A46B 2200/1033; A61Q 5/02; A61Q 19/10; A61Q 3/00; A45D 19/00; A46B 13/008; A46B 13/02; A46B 9/06; A46B 5/0095; A46B 15/0044; A47K 7/04; A46B 15/0006; A61K 2800/87; A45D 2019/0041; A46B 2200/1013; A61K 8/0216

(57) ABSTRACT

A keratinous treatment kit is configured to apply a semi-solid or solid, non-Newtonian cosmetic composition onto a surface of keratinous tissue, wherein the cosmetic kit is packaged in a unitary package. The keratinous treatment kit includes a hybrid applicator, a hand piece, and a semi-solid or solid non-Newtonian cosmetic composition. The hybrid applicator includes a base and cleaning elements, the cleaning elements including a bristled brush and a elastomer contact surface. The handpiece includes a body and a motor, the body configured to receive the hybrid applicator, and the motor arranged and disposed to provide oscillating motion to the hybrid applicator, when attached, relative to the body. The semi-solid or solid, non-Newtonian cosmetic composition is transformable to a spreadable consistency when subjected to oscillating contact with the hybrid applicator. Also provided is a cosmetic application method of utilizing the cosmetic kit.

### FIELD OF THE INVENTION

The present invention is generally directed to a keratinous treatment kit and a method for treating keratinous substrates. More specifically, the present invention is directed to a keratinous treatment kit including a hybrid applicator, a handpiece and a semi-solid or solid non-Newtonian cosmetic composition, and a keratinous treatment method of providing the keratinous treatment kit.

### BACKGROUND OF THE INVENTION

For all those who use facial makeup, particularly on a regular basis, there is the ongoing requirement of effectively removing existing makeup before applying new makeup, both to maintain good skin health and also to facilitate the application of new makeup.

Typically, conventional applicator devices have utilized a liquid cosmetic composition during treatment. The liquid cosmetic composition, however, is not entirely user-friendly. In particular, during handling or traveling, the liquid cosmetic composition has big/bulky volume and is likely to escape or spill the composition inadvertently from a package. In addition, the conventional applicator device has not been sufficiently effective to treat a keratinous substrate.

Therefore, there exists a need to provide a cosmetic kit comprising a semi-solid or solid non-Newtonian cosmetic composition that is configured such that rubbing or movement across the skin will transform the composition into a viscous oil, thereby being more travel-friendly and easier to handle; and an applicator device adopted to capture the semi-solid or solid non-Newtonian cosmetic composition and later transform them into a viscous oil.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

The present teachings contemplate a keratinous treatment kit including a semi-solid or solid non-Newtonian cosmetic composition, a hybrid applicator and a handpiece, and keratinous treatment method of utilizing the keratinous treatment kit.

In an exemplary embodiment, a cosmetic kit for applying a cosmetic composition onto a surface of keratinous tissue is provided. The cosmetic kit, packaged in a unitary package, includes a hybrid applicator, a handpiece, and a semi-solid or solid non-Newtonian cosmetic composition. The hybrid applicator includes a base and cleaning elements. The cleaning elements includes a bristled brush and an elastomer contact surface. The handpiece includes a body and a motor. The body is configured to receive the hybrid applicator, and the motor is arranged and disposed to provide oscillating motion to the hybrid applicator, when attached, relative to the body. The semi-solid or solid non-Newtonian cosmetic composition is transformable to a spreadable consistency when subjected to oscillating contact with the hybrid applicator.

In another exemplary embodiment, a cosmetic kit for applying a cosmetic composition onto a surface of keratinous tissue is provided. The cosmetic kit, packaged in a unitary package, includes a hybrid applicator, a handpiece, and a semi-solid or solid non-Newtonian cosmetic composition. The hybrid applicator includes a base and cleaning elements. The cleaning element includes a bristled brush and an elastomer contact surface. The handpiece includes a body and a motor. The body is configured to receive the hybrid applicator, and the motor is arranged and disposed to provide oscillating motion to the hybrid applicator, when attached, relative to the body. The semi-solid or solid non-Newtonian cosmetic composition, for example, may include anhydrous based systems containing shear sensitive raw materials, aqueous based systems containing shear sensitive raw materials, blends containing shear sensitive raw materials, or combinations thereof. The semi-solid or solid non-Newtonian cosmetic composition is transformable to a spreadable consistency when subjected to oscillating contact with the hybrid applicator.

In yet another exemplary embodiment, a method for cleansing and exfoliating a surface of keratinous tissue includes the step of providing a hybrid applicator comprising a base and cleaning elements comprising a bristled brush and a contact surface. The contact surface may be formed of elastomers including but not limited to TPE, TPU, silicone and combinations thereof. In some particular embodiments, the elastomer is TPE. The method further includes the step of providing a handpiece comprising a body and a motor. The body is configured to receive the hybrid applicator, and the motor is arranged and disposed to provide oscillating motion to the hybrid applicator, when attached, relative to the body. The method further includes the step of applying a semi-solid or solid non-Newtonian cosmetic composition onto the hybrid applicator. The semi-solid or solid non-Newtonian cosmetic composition, for example, may include the group consisting of anhydrous based systems containing shear sensitive raw materials, aqueous based systems containing shear sensitive raw materials, blends containing shear sensitive raw materials or combinations thereof. The method further includes the step of applying the cosmetic composition onto the hybrid applicator. The method further includes the step of actuating the hybrid applicator by controlling a control switch and one of a multimodal switch and knobs to select one of two or more modes of operation. The modes of operation can be selected from at least (i) a cosmetic shear-thinning mode characterized by operation of the hybrid applicator at or above a frequency about 100 Hz with an oscillation amplitude in the range of 3-18° peak-to-peak, the cosmetic shear-thinning mode providing shear-thinning/destructing of the cosmetic composition to a consistency suitable for application, (ii) a cosmetic melting mode characterized by operation of the hybrid applicator at or above a frequency about 175 Hz with an oscillation amplitude in the range of 3-18° peak-to-peak, the cosmetic melting mode providing melting of the cosmetic composition to a consistency suitable for application; and (iii) an application mode characterized by operation alternating between two frequencies 100 Hz and 175 Hz with an oscillation amplitude in the range of 3-18° peak-to-peak, the application mode enabling application to the surface of keratinous tissue.

Other features and advantages of the present invention will be apparent from the following more detailed description of the embodiments which illustrate, by way of example, the principles of the invention.

### BRIEF DESCRIPTION OF THE DRAWINGS

FIG. 1 shows a schematic example of a cosmetic kit.

FIG. 2 shows a perspective view of one example of a hybrid applicator and handpiece.

FIG. 3 shows a top view of an exemplary silicone applicator (left), hybrid applicator (center) and bristled brush (right).

FIG. 4 shows a schematic diagram of one embodiment of an applicator and an accompanying solid or semi-solid non-Newtonian makeup removal member.

FIG. 5 shows a schematic view of another embodiment of the applicator and accompanying solid or semi-solid non-Newtonian makeup removal member.

FIG. 6 shows a schematic view of a still further embodiment of the applicator and accompanying solid or semi-solid non-Newtonian makeup removal member.

FIG. 7 shows a schematic view of yet another embodiment of the applicator and accompanying makeup remover element.

FIG. 8 shows the dynamic viscosity and modulus of semi-solid or solid non-Newtonian cosmetic composition (FLA96100445) as a function of frequency at low strain.

FIG. 9 shows a top view of semi-solid, solid, or non-Newtonian cosmetic composition (FLA96100445) transformed to a viscous oil by oscillation at 175 Hz for 20 seconds.

FIG. 10 shows the comparison of a silicon applicator and a hybrid applicator in terms of effect of shear rate on shear viscosity

FIG. 11 shows the comparison of makeup removal on bioskin using hybrid applicator with monodose cleansing balm (FLA96100445) at 20 HZ with 100% strain.

FIG. 12 shows good melting/shear-thinning of a formula with the added benefit of efficient make-up removal through a wicking/drawing-up effect.

FIG. 13 shows a flow chart diagram illustrating an embodiment of a method, according to an embodiment of the present disclosure.

The exact dimension may vary from dimensions shown in figures, depending upon the nature of application.

Wherever possible, the same reference numbers will be used throughout the drawings to represent the same parts.

### DETAILED DESCRIPTION OF THE INVENTION

The detailed description set forth below in connection with the appended drawings where like numerals reference like elements is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

All numbers expressing quantities of ingredients and/or reaction conditions are to be understood as being modified in all instances by the term “about”, unless otherwise indicated.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The term “applying a cosmetic composition onto a surface of keratinous tissue”, as used herein, and variations of this phrase are intended to mean contacting the fibers, with at least one of the compositions of the invention, in any manner.

The term “at least one,” as used herein, means one or more and thus includes individual components as well as mixtures/combinations.

The term “comprising” (and its grammatical variations), as used herein, is used in the inclusive sense of “having” or “including” and not in the exclusive sense of “consisting only of.”

The term “keratinous substrate”, as used herein, includes, but is not limited to, skin, nails or hair. “Keratinous substrate” also includes “keratinous tissue” or “keratinous fibers,” which as defined herein, may be human keratinous fibers, and may be chosen from, for example, hair, such as hair on the human head, or hair comprising of eyelashes or hair on the body.

The term “treat” (and its grammatical variations), as used herein, refers to the application of the compositions of the present invention onto keratinous substrates such as keratinous fibers or skin.

The term “semi-solid” and variations of this phrase, as used herein, refer to a highly viscous substance that lies along the boundary between solid and liquid.

The term “unidoses” and variations of this phrase, as used herein, refer to a singular composition or substance that does not require secondary counterparts.

The term “shear sensitive raw materials”, as used herein, refers to substances sensitive to shears.

The term “shear thinning/destructuring” and variations of this phrase, as used herein, refer to lowering the viscosity of the composition as shears applied to the composition increases.

The term “melting”, as used herein, refers to a phase transition of a substance from a solid to liquid.

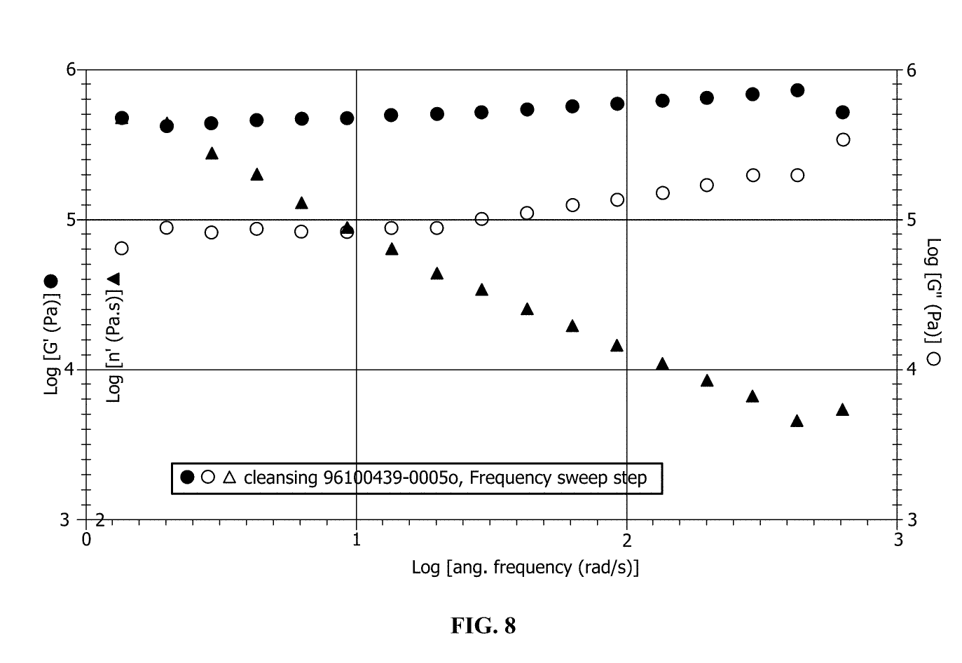

Referring to FIG. 8, inventors discovered that the cosmetic kit and method, according to the present disclosure, activated accelerated transformation of a high viscosity semi-solid or solid non-Newtonian composition to a less viscous matter through the device's high frequency oscillating energy when the consumer is ready to use it. This finding enables customers to provide good spreading and deposition via shear thinning/destructuring upon use with the cosmetic kit by delivering a less viscous composition as unidoses for cosmetic purposes.

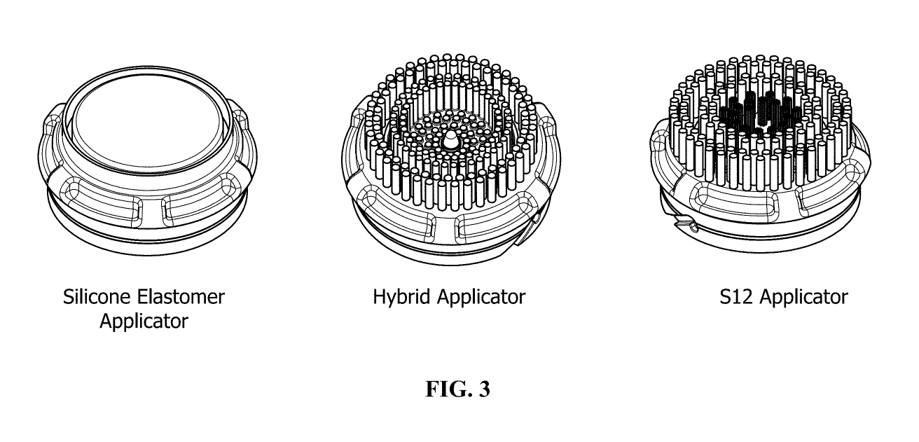

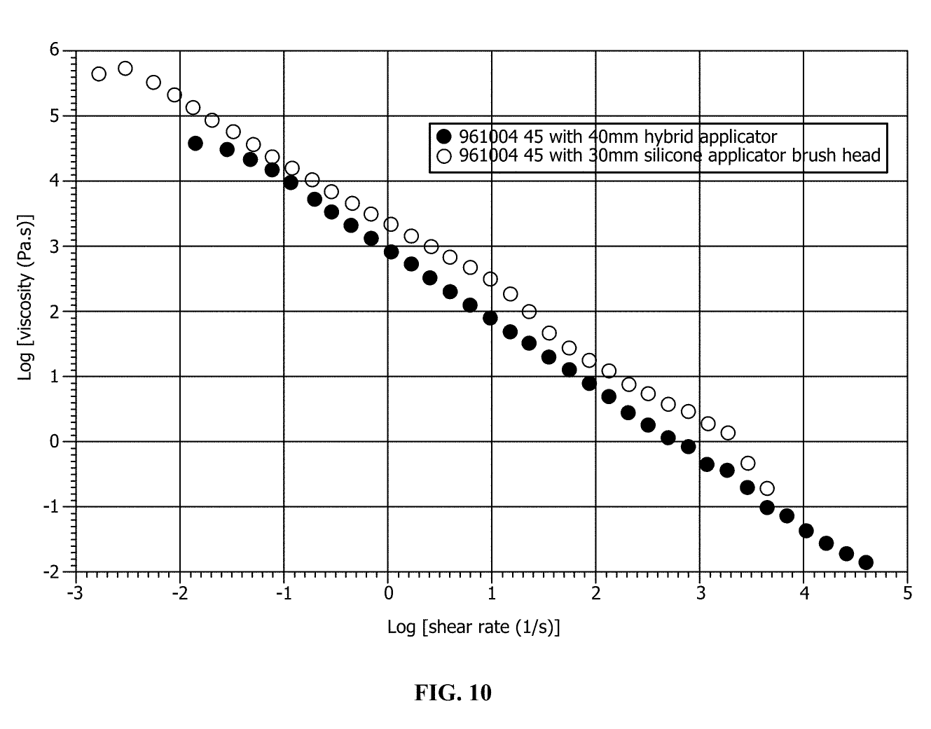

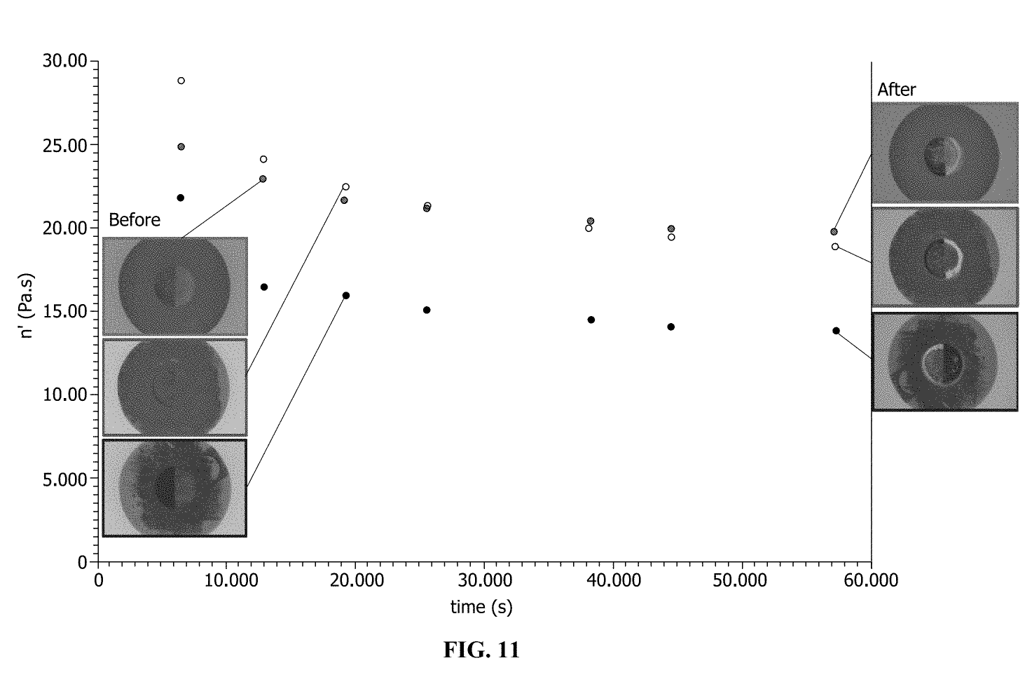

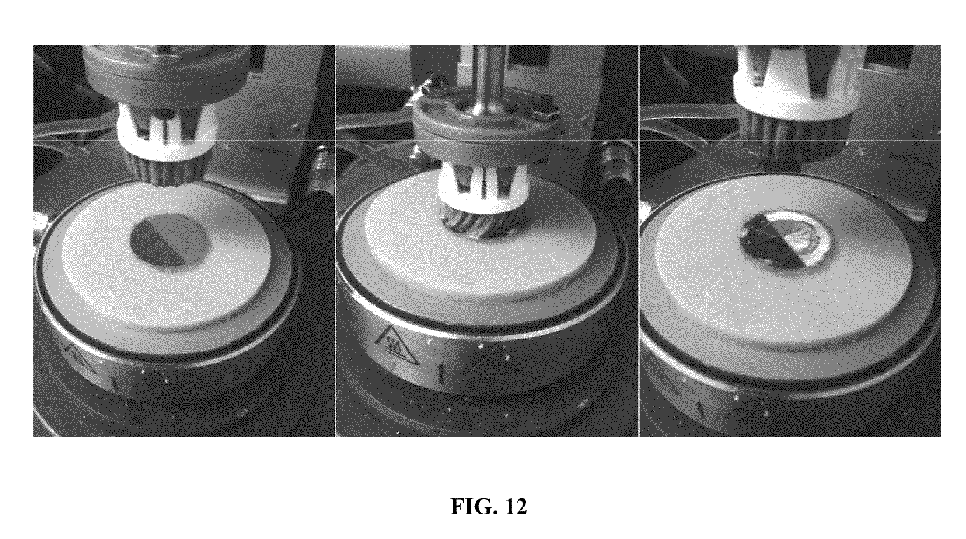

In addition, referring to FIG. 10-12, it was surprisingly discovered by the inventors that the hybrid applicator comprising a bristled brush and elastomers including but not limited to TPE, TPU, silicone and combinations thereof shows superior performance compared to other applicators including an applicator comprising silicone elastomer and an applicator comprising a bristled brush, as shown in FIG. 3, in both texture transformation and makeup removal performance upon use with the semi-solid or solid non-Newtonian cosmetic composition.

It was further unexpectedly discovered by the inventors that the speed of rotation needed to achieve a shear thinning effect would be so high that it would twist the skin, resulting in too much friction that would scratch the skin and lead to irritation. This discovery is important because it demonstrates that the device's oscillation and amplitude settings without rotation, according to the present disclosure, can uniquely deliver a shear-thinning/destructuring benefit in a first mode; and upon actuation to a second mode, can safely oscillate when in contact with skin to provide even application of the cosmetic composition, thus providing a competitive advantage over devices using rotational motions.

Hybrid Applicator Device

In some embodiments, the motor may present an electric drive motor assembly. The drive motor assembly in an embodiment may include a power storage source, a drive control, a drive shaft, and a drive motor. The drive control and drive motor may be powered by the power storage source. The motor, according to the invention, may not deliver rotational and vibrational motions in an exemplary embodiment. The motor may deliver only an oscillating motion via the drive shaft to the brush or applicator in the exemplary embodiment. The motor transmits oscillating motion to the hybrid applicator through the body of the handpiece, when the hybrid applicator is attached to the body of the handpiece. In accordance with the descriptions herein of representative embodiments, when referred to, the degrees of motion and/or amplitude are with respect to peak-to-peak oscillating motions.

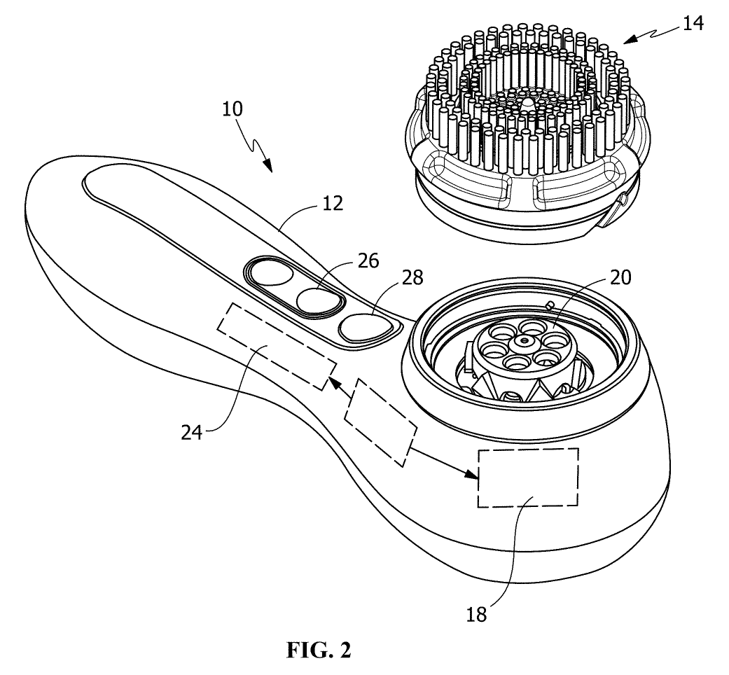

Referring to FIG. 2, a handpiece 10 includes a body. In an embodiment, an applicator 14 is configured and adapted for makeup removal when the appliance is operated as described below. The body 12 shown is for illustration purposes only. Various handles can be used with the applicator shown and described herein. Body 12 includes a drive assembly which includes a motor 18 which operates on a drive member 20 to which the applicator 14 is attachable. The applicator 14 can easily be fitted to and removed from the drive member and the handle. The motor 18 is powered by a rechargeable battery 22 and controlled by a microprocessor 24. The microprocessor can be programmed to provide various frequencies and amplitudes for movement of the applicator in an oscillatory manner. Depending on the use, for example, application of makeup removal, particular combinations of frequency and amplitude can be selected, as further described herein. The handle may include a multimodal switch 28 for control and operation of the appliance as well as control switch 26 for control of power increase and decrease. In other embodiments, the multimodal switch 28 may have multiple operational modes. For example, the switch 28 may include three operational modes: initiation, transformation, and application modes. The switch 28 may include a built-in illuminating indicator including but not limited to LCD, LED and OLED to indicate the operational modes. A user may press the multimodal switch 28 for predetermined time (e.g. three seconds) to turn on and initiate the hybrid applicator device. The built-in illuminating indicator may flash blue while the hybrid applicator device is being initiated. Once initiation is completed, the built-in illuminating indicator may change from flashing blue to solid blue to notify users that the device is ready for transformation.

A user may subsequently apply a semi-solid or solid non-Newtonian cosmetic composition onto applicator 14. A user, then, may press the switch 28 once more to start transformation of the cosmetic composition to a spreadable consistency. The spreadable consistency may include but not be limited to viscous oil. The transformation mode may further include two modes of operation: a cosmetic shear-thinning mode and a cosmetic melting mode. The shear-thinning mode may drive the applicator with an oscillating motion at or above a frequency about 100 Hz with an oscillation amplitude in the range of 3-18° peak-to-peak. Similarly, the cosmetic melting mode may drive the applicator with an oscillating motion at or above a frequency about 175 Hz with an oscillation amplitude in the range of 3-18° peak-to-peak. A user may transition between the cosmetic shear-thinning mode and the cosmetic melting mode using the control switch 26. The oscillating motion may provide complete transformation of the cosmetic composition to a spreadable consistency. While being transformed to a spreadable consistency either by the cosmetic shear-thinning mode or the cosmetic melting mode, the built-in illuminating indicator may emit red light. Once transformation is completed, the built-in illuminating indicator may change from red to green to notify users that the device is ready for application. A user, then, may press the switch 28 to apply the transformed cosmetic composition to a surface of keratinous tissue. In some embodiments, the application mode may drive the applicator with oscillating motions alternating between two frequencies. In other embodiments, the application mode may drive the applicator with oscillating motions alternating between frequencies 100 Hz and 175 Hz or other frequency combinations that are complimentary to the resonance characteristics of the motor drive 18 system with an oscillation amplitude in the range of 3-18° peak-to-peak. In yet other embodiments, the application mode may drive the applicator with oscillating motions alternating between one frequency at or above 100 Hz and the other frequency at or above 175 Hz with an oscillation amplitude in the range of 3-18° peak-to-peak. In yet other embodiments, the application mode may drive the applicator with oscillating motions at a frequency between 100 Hz and 175 Hz with an oscillation amplitude in the range of 3-18° peak-to-peak. In yet other embodiments, the application mode may drive the applicator with oscillating motions at a frequency at or below 100 Hz with an oscillation amplitude in the range of 3-18° peak-to-peak to minimize potential damage to the skin. During the application mode, a user may press the switch 28 to stop application temporarily. A user may press the switch 28 again to renew the application. In some embodiments, the application mode may deliver the applicator with oscillating motions for pre-determined time. Once application is finished, a user may manually press the switch 28 for predetermined time (e.g. three seconds) in order to turn off the device completely. The built-in illuminating indicator may be turned off as well to notify the user that the device is turned off.

In alternative embodiments, the handpiece 10 may include knobs in addition to multimodal switch 28. In other embodiments, the handpiece 10 may include knobs instead of multimodal switch. The knobs may have multiple operational modes. For example, the knobs may have three operational modes: initiation, transformation, and application modes. A user may easily rotate the knobs to switch among the three operational modes. In the beginning, a user may press the switch 28 for predetermined time (e.g. three seconds) to turn on the hybrid applicator device. A user may initiate the hybrid applicator device by rotating the knobs. Once initiation is completed, a user may subsequently apply a semi-solid or solid non-Newtonian cosmetic composition onto applicator 14. A user then may rotate the knobs to start the transformation of the cosmetic composition to a spreadable consistency. The transformation mode may further include two modes of operation: a cosmetic shear-thinning mode and a cosmetic melting mode. The shear-thinning mode may drive the applicator with an oscillating motion at or above a frequency about 100 Hz with an oscillation amplitude in the range of 3-18° peak-to-peak. Similarly, the cosmetic melting mode may drive the applicator with an oscillating motion at or above a frequency about 175 Hz with an oscillation amplitude in the range of 3-18° peak-to-peak. In general, as amplitude and frequency increase, the melting effect is improved. A user may transition between the cosmetic shear-thinning mode and the cosmetic melting mode using the control switch 26. The knobs may further adjust the oscillation amplitude at users' preferences. The oscillating motion may provide complete transformation of the cosmetic composition to a spreadable consistency. A user, then, may press rotate the knobs to apply the transformed cosmetic composition to a surface of keratinous tissue. In some embodiments, the application mode may drive the applicator with oscillating motions alternating between two frequencies. In other embodiments, the application mode may drive the applicator with oscillating motions alternating between frequencies 100 Hz and 175 Hz with an oscillation amplitude in the range of 3-18° peak-to-peak. In yet other embodiments, the application mode may drive the applicator with oscillating motions alternating between one frequency at or above 100 Hz and the other frequency at or above 175 Hz with an oscillation amplitude in the range of 3-18° peak-to-peak. In yet other embodiments, the application mode may drive the applicator with oscillating motions at a frequency between 100 Hz and 175 Hz with an oscillation amplitude in the range of 3-18° peak-to-peak. In yet other embodiments, the application mode may drive the applicator with oscillating motions at a frequency at or below 100 Hz with an oscillation amplitude in the range of 3-18° peak-to-peak to minimize potential damage to the skin. During the application mode, a user may press the switch 28 to stop application temporarily. A user may press the switch 28 again to renew the application. In some embodiments, the application mode may deliver the applicator with oscillating motions for pre-determined time. Once application is finished, a user may manually press the switch 28 for predetermined time (e.g. three seconds) in order to turn off the device completely. The built-in illuminating indicator may be turned off as well to notify the user that the device is turned off.

In other embodiments, the control switch 26 may have two buttons: an upper push button and a lower push button. During the transformation or application, a user may press the upper push button to increase the frequency of an oscillating motion. Similarly, a user may press the lower push button to decrease the frequency of an oscillating motion.

In some embodiments, other computer intelligence (i.e. RFID, NFC, Bluetooth) may be used to change internal settings including multiple operational modes. In embodiments with RFID communication between brush heads equipped with RFID tags and the handle can result in the microprocessor 24 set-up to enable during the use protocol a mix of frequencies and amplitudes that provide improved shear thinning characteristics of a particular formula or improved user perception. With RFID controls, the applicator may be driven with an oscillating motion at a frequency within the range of about 60-300 Hz and at any suitable amplitude between 3-18° peak-to-peak all in the same use period.

In some embodiments, the applicator 14 may be used with a Newtonian cosmetic composition as well. The Newtonian cosmetic composition including but not limited to liquid or soft formulations (oil, emulsions) or powder (Newtonian solid) could be used with the hybrid applicator in such a way that the applicator depression serves a fill line for dosing and allows more accurate product use.

FIGS. 2-7 show embodiments of the makeup removal applicator, referred to hereinafter as the applicator. The embodiments of FIGS. 2-7 are representative of a variety of applicator arrangements which are possible relative to providing, among other things, effective makeup removal when used with an appliance having the operational characteristics described below. The applicator is used with a non-Newtonian solid, semi-solid, gel or the like dose/tablet of a balm that in liquid form is effective in removing makeup from the skin. In the embodiment shown, the applicator may be driven with an oscillating motion at a frequency within the range of about 60-300 Hz. Thus in various embodiments, the applicator may be driven with an oscillating motion at a frequency from about 60, 70, 80, 90, 100, 110, 120, 130, 140, 150, 160, 170, 180, 190, 200, 210, 220, 230, 240, 250, 260, 270, 280, 290 to about 300 Hz, including increments and intervals therein. In some embodiments, the applicator may be driven with an oscillating motion at or above a frequency about 100 Hz. In other embodiments, the applicator may be driven with an oscillating motion at or above a frequency about 150 Hz. The oscillating motion may have an oscillation amplitude in the range of 3-18° peak-to-peak. The exemplary applicator may be driven with an oscillating motion between 150-186 Hz. Thus in various embodiments, the oscillating motion may have an oscillation amplitude from about 3, 4, 5, 6, 7, 8, 9, 10, 11, 12, 13, 14, 15, 16, 17 to about 18° peak-to-peak, including increments and intervals therein. In some embodiments, the oscillating motion may have an oscillation amplitude about 9° tangential.

The oscillating motion of the hybrid applicator at or above a frequency higher than 100 Hz with an amplitude in the range of 3-18° peak-to-peak may provide shear-thinning/destructuring of the semi-solid or solid non-Newtonian cosmetic composition.

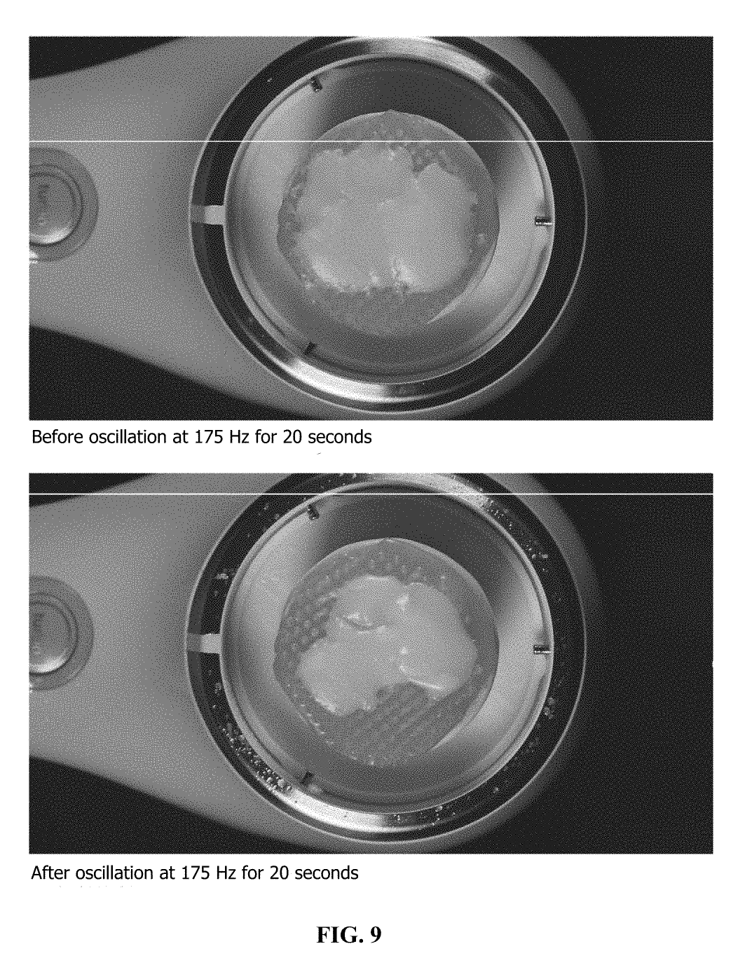

Referring to FIG. 9, the oscillating motion of the hybrid applicator at a frequency 175 Hz with an amplitude in the range of 3-18° peak-to-peak may provide melting of the semi-solid or solid non-Newtonian cosmetic composition.

In some embodiments, the oscillating motion of the hybrid applicator at a frequency higher than a frequency f₁ with an amplitude in the range of 3-18° peak-to-peak may provide shear-thinning/destructuring of the semi-solid or solid non-Newtonian cosmetic composition. The semi-solid or solid non-Newtonian cosmetic composition, for example, may include anhydrous based systems containing shear sensitive raw materials, aqueous based systems containing shear sensitive raw materials, blends containing shear sensitive raw materials or combinations thereof.

In some embodiments, the oscillating motion of the hybrid applicator at a frequency higher than a frequency f₂ within a range of amplitude of with an amplitude in the range of 3-18° peak-to-peak may provide melting of the semi-solid or solid non-Newtonian cosmetic composition. The semi-solid or solid non-Newtonian cosmetic composition, for example, may include anhydrous based systems containing shear sensitive raw materials, aqueous based systems containing shear sensitive raw materials, blends containing shear sensitive raw materials, and combinations thereof.

In operation, with each of the embodiments, kinetic energy from the oscillating motor is coupled into the brush, which is oscillating at the above-noted frequency and amplitude. In an embodiment, the non-Newtonian solid, semi-solid, gel or the like dose of a formulation reversibly transforms from a solid, semi-solid, gel or the like in the presence of an applied energy stimulus. This transformation enables customers to provide good spreading and deposition via shear-thinning/destructuring upon use with the cosmetic kit by delivering a less viscous composition as unidoses for cosmetic purposes. In an embodiment, once the applied energy stimulus is stopped, the non-Newtonian solid, semi-solid, gel or the like dose returns to its solid, semi-solid, gel or the like state. In an embodiment, the non-Newtonian solid, semi-solid, gel or the like dose of a formulation reversibly transforms from a solid, semi-solid, gel or the like to a liquid by the kinetic energy coupled to the brush, with the liquid then applied to the human skin for removal of makeup, application of a cosmetic coating and the like. The non-Newtonian shear-thinning material changes state from a solid, semi-solid, gel or the like to a liquid in the presence of an applied energy stimulus, and changes back to a solid, semi-solid, gel or the like in the absence of the applied energy stimulus. A shear-thinning material by definition is one in which when an applied shear stress is increased, the material's viscosity decreases in a nonlinear way. This results in the material changing its state from a solid, semi-solid, gel or the like to a liquid. In the present case, this permits a solid, semi-solid, gel or the like tablet of balm to be initially conveniently and reliably anchored or staked to the applicator. The appliance with the applicator can then be elevated to a vertical position adjacent the skin without the solid, semi-solid, gel or the like tablet falling out or a liquid spilling away. It is advantageous for users to apply cosmetics in a controlled manner upon use with the appliance.

The coupling of the kinetic energy from the motor to the applicator and to the solid, semi-solid, gel or the like balm tablet can be accomplished in a number of ways with the present applicator. First, the applicator in one or more embodiments has filaments or other flexible members such that when the applicator is oscillated, the filaments will flex and move, rubbing against the non-Newtonian solid, semi-solid, gel or the like balm tablets staked in the applicator. Further, kinetic energy may be coupled directly to the applicator through the use of a stake or stakes or other elements fixedly contacting the solid, semi-solid, gel or the like balm element. Still further, the solid, semi-solid, gel or the like balm element can be staked or held in such a way in the applicator that when the applicator oscillates, the balm tablet contacts a region of the skin and by virtue of the shear forces in that plane, the non-Newtonian solid, semi-solid, gel or the like dose changes to a liquid for effective removal of makeup.

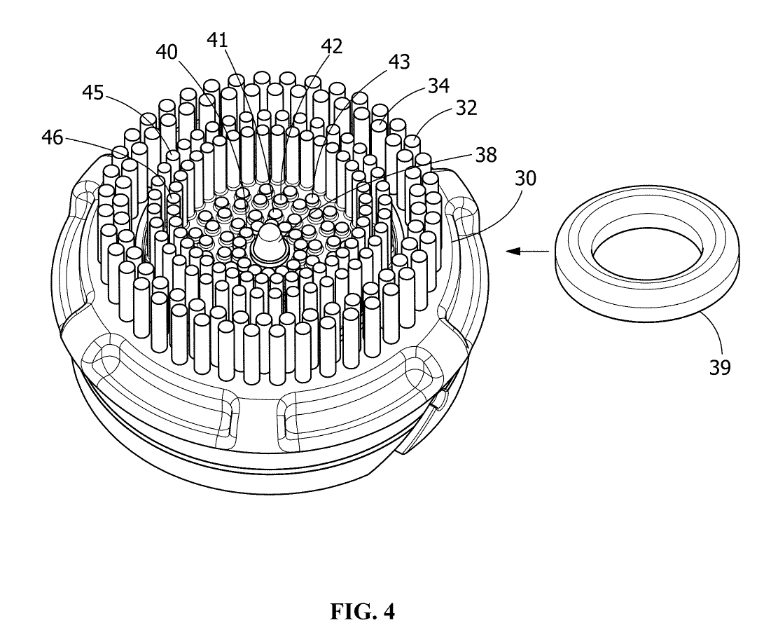

One embodiment is shown in FIG. 4. It includes an applicator base 30 which is configured and adapted to mate with the drive member in the handle as shown in FIG. 2. One arrangement for such a connection is shown in U.S. Pat. No. No. 7,386,906, which is owned by the assignee of the present invention and which are both hereby incorporated by reference. The base 30 is fixed and does not oscillate in operation of the appliance. The embodiment of FIG. 4 includes two outer rings of tufts 32 and 34 of bristles. In the embodiment shown, the two rings of tufts are mounted in the base 30, and hence do not move in operation of the appliance. They are constructed with 3-4 mil soft filaments including but not limited to single and double tapered PBT filaments cut to a height between 0.455 inches to 0.500 inches, end-rounded and polished, with normal filament packing in each tuft. These two rings of tufts form the outer boundaries of the applicator and serve, among other purposes, as a protective shield against splatter, and also assist in lifting off makeup. In some other embodiments, the soft filaments may include but not be limited to DuPont supersoft filaments with a trim height of 0.425 inches, nylon, PET, PBT (polyester) elastomeric filaments, including supersoft/Hytrel™ cellulose, polycarbonate, etc.

In an embodiment, the inner portion of the brush includes at its center a stake element 38 which in the embodiment shown is a plastic silicon part with a Shore hardness of 40 A in the embodiment shown. The stake 38 is approximately 0.2 inches high and is approximately conical in shape. Stake 38 serves as an anchoring member for the non-Newtonian dose element 39. In an embodiment, the stake 38 includes symmetrical or non-symmetrical structures to further help anchor a non-Newtonian solid, semi-solid, gel or the like tablet. In an embodiment, one or more regions of the inner portion include tufts or bristles of elastomeric protrusions arranged in regular or irregular patterns. In some other embodiments, the applicator optionally may not contain the stake element 38.

Surrounding the stake 38 in the embodiment shown in FIG. 4 are four rings of silicon protrusion elements 40-43 that will vary in height from embodiment to embodiment from 0.05 inches to 0.35 inches, with a diameter of 0.06 inches. These protrusion elements may be formed of an elastomer including but not limited to; TPE, TPU, silicone, and combinations thereof. Alternatively, this could be any other form of plastic. The stake element will always be taller than the rings of protrusions. The spacing of the rings of protrusions is approximately between 0.06 inches and 0.09 inches. In any one embodiment, the height of the elastomeric protrusions will be approximately the same. The dimensions can vary to some extent. The lower surface of the balm dose 39 will contact the ring's elastomeric protrusions. The remainder of the inner applicator portion comprises two rings 45 and 46 of filaments, although one or more additional rings of filaments can also be used. In some embodiments, the filaments of two rings 45 and 46 may or may not have the same height as the filaments of other two rings 34 and 32. In some other embodiments, the filaments of two rings 45 and 46 may or may not have the same height as the filaments 43. These rings have a height of 0.425 inches and are comprised in the embodiment shown of 3-4 mil soft filaments including but not limited to single and double tapered PBT filaments cut to a height between 0.455 inches to 0.500 inches, which are end-rounded and polished, to permit a gentle interaction with the skin. The soft filaments are used for its ability to pick up and remove makeup as well as being comfortable for the user. In some other embodiments, the soft filaments may include but not be limited to DuPont supersoft filaments with a trim height of 0.425 inches, nylon, PET, PBT (polyester) elastomeric filaments, including supersoft/Hytrel™, cellulose, polycarbonate, etc.

Materials of different stiffness in the driven applicator can cause an out-of-phase motion between those materials. In the case of the present applicator, the motion is approximately 140°-180° out-of-phase between the innermost ring 46 of soft filaments including but not limited to single and double tapered PBT filaments, DuPont supersoft filaments, nylon, PET, PBT (polyester) elastomeric filaments, including supersoft/Hytrel™, cellulose, polycarbonate, etc.

Supersoft filaments in the inner applicator portion and the outermost (fourth) row 43 of elastomeric protrusions. Thus in various embodiments, the motion may be approximately from 140, 150, 160, 170 to about 180°, including increments and intervals therein. When the loaded inner applicator portion moves at 175 Hz with an amplitude in the range of 4-9°, with radii of the two above elements at 0.335 inches and 0.375 inches, respectively, directional velocities of 8.03-18.50 inches per second and 9.08-20.56 inches per second result. Since the above two elements of the inner applicator are moving, respectively, within the two velocity ranges described above, 140°-180° out-of-phase, the relative velocity between the two rings of elements is much higher than that indicated above. The relative velocity, i.e. the sum of the two individual velocities, that the solid, semi-solid, gel or the like balm tablet would experience would range from 7.06 inches per second to 39.06 inches per second. Within this range of relative velocity, the balm tablet shear thins from solid, semi-solid, gel or the like to a liquid under normal loading conditions. The liquid makeup remover formulation, with the oscillating action of the inner portion of the applicator, operates to effectively and quickly remove facial makeup.

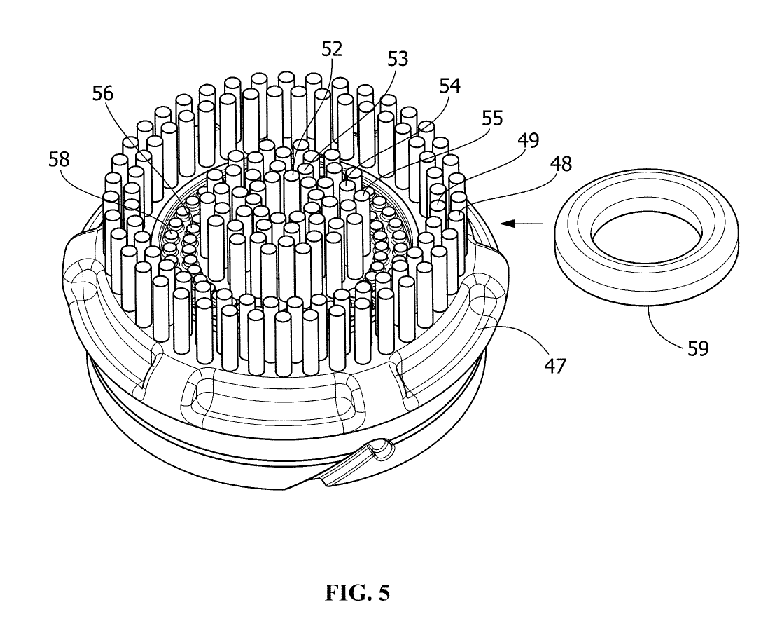

A second embodiment is shown in FIG. 5. This embodiment is similar to the embodiment of FIG. 4, in that it includes the same applicator base 47 and two fixed outer rings 48 and 49, with similar material and sizing as the embodiment of FIG. 4. The oscillating inner applicator portion includes a center portion of filament rings, comprising filaments which are similar to the filaments comprising rings 44 and 46. These filaments form four concentric rings 52-55. The outside diameter of ring 55 in the embodiment shown is approximately 0.84 inches. Positioned between the center portion filament rings 52-55 and the outer fixed rings 48 and 49 are two rings of elastomeric protrusions 56 and 58. In some embodiments, the filaments of two rings 56 and 58 may or may not have the same height as the filaments of other two rings 48 and 49. In some other embodiments, the filaments of two rings 56 and 58 may or may not have the same height as the filaments 52. These protrusions may be formed of elastomers including but not limited to TPE, TPU, silicone and combinations thereof. Alternatively, this could be any other form of plastic. These protrusions are approximately 0.06 inches high and have a diameter of approximately 0.06 inches. The non-Newtonian balm tablet 59 associated with this embodiment is doughnut-shaped, having an inner diameter and an outer diameter such that the tablet fits within the area between the center portion of filament rings and the fixed outer rings of filaments, contacting the intermediate rings of protrusions. The doughnut tablet is configured such that the sides of the outer row 55 of filaments holds the tablet in place when the appliance is lifted to a position where the applicator contacts the skin. In operation, the shear thinning effect changes the tablet from a solid, semi-solid, gel or the like balm tablet to a liquid useful in makeup removal, by virtue of the various shear stress effects discussed above, including the differential motion effect described above with respect to the embodiment of FIG. 4.

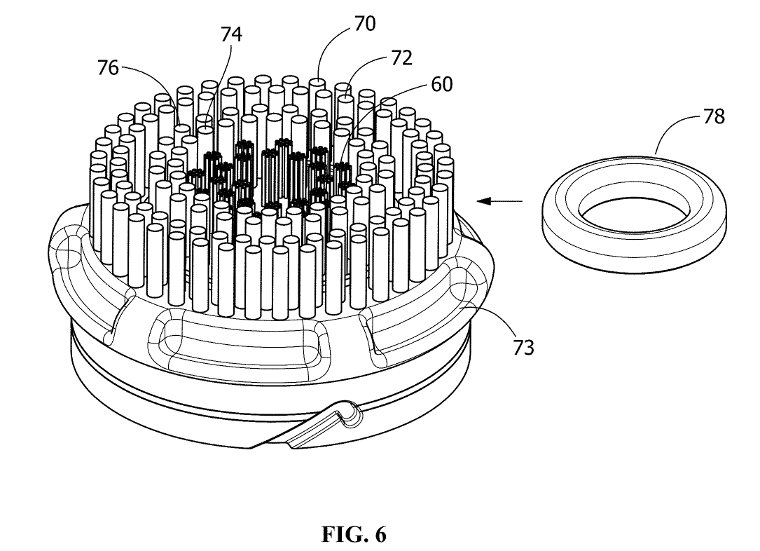

FIG. 6 shows a further embodiment, in which the applicator comprises all bristles, instead of including an elastomeric part of the inner applicator (oscillating) portion. This embodiment includes an inner oscillating portion with a center portion 60 of four inner rings of 3-12 mil soft filaments including but not limited to single and double tapered PBT filaments cut to a height between 0.455 inches to 0.500 inches. The height of the single and double tapered PBT filaments easily enables a monodose size that consumers valued and preferred. In some other embodiments, the soft filaments may include but not be limited to DuPont supersoft filaments, nylon, PET, PBT (polyester) elastomeric filaments, including supersoft/Hytrel™, cellulose, polycarbonate, etc. In various embodiments, the soft filaments may be from about 3, 4, 5, 6, 7, 8, 9, 10, 11 to about 12 mil, including increments and intervals therein. The four rings of filaments will generally provide enough stiffness to properly seat and hold the solid, semi-solid, gel or the like balm tablet similar to that provided by the elastomeric protrusions of FIG. 5. The inner brush portion also includes two rings of outer filaments 74, 76 which are taller than the center portion rings 60. This embodiment also includes, similar to the embodiment of FIGS. 4 and 5, two outer rings 70, 72 which are fixed in position in applicator base 73. In some embodiments, the filaments of two rings 74 and 76 may or may not have the same height as the filaments of other two rings 70 and 72. In some other embodiments, the filaments of two rings 74 and 76 may or may not have the same height as the filaments 60. Out-of-phase motion can be achieved between the outer ring of the center portion bristles and the inner ring 74 of the outer bristles of the inner applicator portion to maintain a substantial differential velocity. In some arrangements, the center portion bristles 60 of the oscillating inner portion are 0.012 inch PBT filament bristles in a staggered height varying from 0.240 to 0.280 inches, providing both stiffness to seat the balm tablet 78 as well as the out-of-phase motion to assist in shear thinning, as described above. In this embodiment, the two outer rings of the oscillating inner portion and the two outer fixed rings are still 3 mil soft filaments including but not limited to single and double tapered PBT filaments cut to a height between 0.455 inches to 0.500 inches, DuPont supersoft filaments with a trim height of 0.425 inches, nylon, PET, PBT (polyester) elastomeric filaments, including supersoft/Hytrel™, cellulose, polycarbonate, etc.

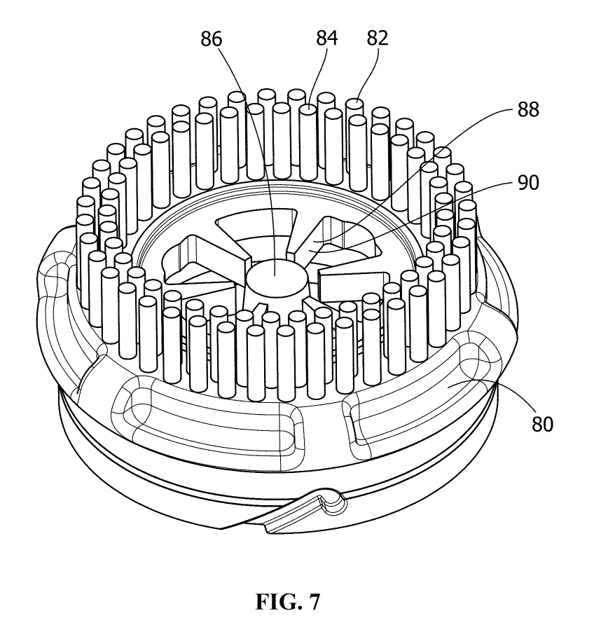

FIG. 7 shows yet another embodiment illustrating a different oscillating inner applicator portion. The embodiment includes a base element 80 and two outer rings 82 and 84 which are fixed in position, having the same configuration and material as the base element and the two outer fixed rings for the other embodiments. The inner applicator portion, however, does not include elastomeric elements or bristles, but rather comprises a wheel-like arrangement with a central inner hub 86, an outer hub 88 and a plurality of spaced vanes 90 which extend from the outer hub in the direction of the central hub, terminating a short distance (almost touching) therefrom. In the embodiment shown, the central hub is approximately 3/16-inch high, approximately ¼-inch in diameter and slightly conical. The outer hub is approximately 3/16-inch high and approximately ⅛-inch thick, with an outside diameter of 1⅛ inches. In the embodiment shown, there are six equally spaced vanes, but this number could vary. The vanes are slightly tapered from the outer hub 88 to their termination point adjacent the inner hub 86. The vanes in particular are made of a flexible plastic, such as silicone TPE, so that when the inner applicator portion oscillates, the vanes also oscillate, rubbing against the lower surface of the solid, semi-solid, gel or the like balm tablet, so that energy from the oscillating action is coupled into the tablet, producing sufficient shear so that the non-Newtonian balm tablet changes from a solid, semi-solid, gel or the like to a liquid for subsequent application to the skin. In an embodiment, a semi-solid non-Newtonian balm is partially anchored by the vanes. In an embodiment, the vanes provide a mechanical strain to a semi-solid non-Newtonian balm so as to cause a change in state. There are openings being adjacent vanes so that the tablet can be pressed against the vanes and slightly into the openings, holding it in place during movement of the appliance from the horizontal position to the operational position against the skin for use.

The bristled brush, according to the invention, may be synthetic, natural, or combinations thereof. It may be, for example, vegetable or natural fibers, metal such as steel, glass, wood, elastically deformable materials such as vulcanized elastomers or thermoplastic materials or combinations thereof.

Slight variation in bristle heights of the bristled brush can enhance the exfoliation of a surface of keratinous tissue from the epidermis.

The TPE, for example, may include Styrenic block copolymers (TPE-s), Thermoplastic olefins (TPE-o), Elastomeric alloys (TPE-v or TPV), Thermoplastic polyurethanes (TPU), Thermoplastic copolyester, Thermoplastic polyamides, Santoprene (ExxonMobil), Termoton (Termopol Polimer), Arnitel (DSM), Solprene (Dynasol), Engage (Dow Chemical), Hytrel (DuPont), Dryflex and Mediprene (ELASTO), Kraton (Kraton Polymers), Pibiflex, Forprene, Termoton-v, Sofprene (SBS) and Larprene (SEBS).

Semi-Solid or Solid Non-Newtonian Cosmetic Composition

Referring to FIG. 8, the semi-solid or solid, non-Newtonian composition may have a shear-thinning nature or behavior. That is, the viscosity of the composition can be reversibly lowered when increasing shears are applied to the composition.

Compositions

The compositions of the present disclosure are generally anhydrous compositions, such that they contain little to no water (e.g., less than 1% by weight water, relative to the total weight of the compositions). The compositions include at least one oil phase and at least one non-ionic surfactant. The oil phase includes one or more linear (i.e., unbranched) fatty esters, such as cetyl palmitate, which can assist in providing a molded shape to the compositions and/or provide skin benefits. In some embodiments, the oil phase can further include at least one branched fatty esters, such as ethylhexyl palmitate and/or isocetyl stearate, which can assist in providing skin cleansing properties, good skin feel, and skin care benefits.

In some embodiments, the compositions of the present disclosure include about 70% to about 95% (e.g., about 70%-about 85%, about 70% to about 80%, about 75% to about 90%, about 80% to about 90%, about 80% to about 95%, or about 90% to about 95%) by weight of the oil phase, of which about 1% to about 30% by weight (e.g., about 10% to about 25% by weight) is the at least one linear (i.e., unbranched) fatty ester; and about 4% to about 8% (e.g., about 4% to about 6%, about 5% to about 8%, about 6% to about 8%) by weight of the at least one non-ionic surfactant, where the weight percentages are relative to the total weight of the compositions. Thus in various embodiments, the compositions of the present disclosure include from about 70, 71, 72, 73, 74, 75, 76, 77, 78, 79, 80, 81, 82, 83, 84, 85, 86,87, 88, 89, 90, 91, 92, 93, 94 to about 95% by weight of the oil phase, including increments and intervals therein, from about 1, 2, 3, 4, 5, 6, 7, 8, 9, 10, 11, 12, 13, 14, 15, 16, 17, 18, 19, 20, 21, 22, 23, 24, 25, 26, 27, 28, 29 to about 30% by weight of at least one linear (i.e., unbranched) fatty ester, including increments and intervals therein; and from about 4, 5, 6, 7 to 8% by weight of the at least one non-ionic surfactant, including increments and intervals therein, where the weight percentages are relative to the total weight of the compositions.

In some embodiments, the at least one linear fatty ester is present in a given composition in an amount of about 1% to about 30% by weight (e.g., about 10% to 25% by weight), relative to the total weight of the composition. For example, the at least one linear fatty ester can be present in an amount of about 1% or more (e.g., about 5% or more, about 10% or more, about 20% or more, or about 25% or more) and/or about 30% or less (e.g., about 25% or less, about 20% or less, about 10% or less, or about 5% or less) by weight in the composition, relative to the total weight of the composition. Thus in various embodiments, the at least one linear fatty ester is present in a given composition in an amount from about 1, 2, 3, 4, 5, 6, 7, 8, 9, 10, 11, 12, 13, 14, 15, 16, 17, 18, 19, 20, 21, 22, 23, 24, 25, 26, 27, 28, 29 to about 30% by weight, including increments and intervals therein, relative to the total weight of the composition.

In some embodiments, the at least one linear fatty acid is cetyl palmitate.

In some embodiments, the at least one branched fatty ester is present in a given composition in an amount of about 1% to about 60% (e.g., about 20% to about 60%, about 25% to about 55%, about 1 to about 40%, or about 5% to about 35%) by weight, relative to the total weight of the composition. For example, the at least one branched fatty ester is present in a given composition in an amount of about 1% or more (e.g., about 5% or more, about 20% or more, about 25% or more, about 35% or more, or about 45% or more) and/or about 60% or less (e.g., about 45% or less, about 35% or less, about 25% or less, about 20% or less, or about 5% or less) by weight, relative to the total weight of the composition. Thus in various embodiments, the at least one branched fatty ester is present in a given composition in an amount from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 to about 60% by weight, including increments and intervals therein, relative to the total weight of the composition.

In some embodiments, the at least one branched fatty ester is ethylhexyl palmitate and/or isocetyl stearate.

In some embodiments, the compositions do not contain paraffin wax, mineral oil, or an anionic surfactant, or any combination thereof.

Oil Phase

As described above, the compositions of the present disclosure include an oil phase. The oil phase can include three major groups of compounds: oils (which include fats and butters), esters, and waxes. In some embodiments, in addition to the at least one linear fatty ester in the oil phase of the compositions, the oil phase further includes an oil, a wax, an ester, or any combination thereof. In some embodiments, in addition to the at least one linear fatty ester in the oil phase of the compositions, the oil phase further includes an oil, a wax, an ester, or any combination thereof; the ester can be other than a linear fatty ester. In certain embodiments, in addition to the at least one linear fatty ester in the oil phase of the compositions, the oil phase further includes an oil, a wax, an ester, or any combination thereof; the ester can be other than a linear or branched fatty ester. The oil phase can provide emollient properties to the compositions.

Oils

In some embodiments, when the oil phase further includes an oil, the oil is present in an amount of about 0.5% to about 20% (e.g., about 1% to about 20%, about 1% to about 15%, about 5% to about 15%, about 5% to about 10%, about 0.5% to about 5%, or about 0.5% to about 2%) by weight, relative to the total weight of the composition. For example, the oil can be present in a given composition in an amount of about 0.5% or more (e.g., about 1% or more, about 2% or more, about 5% or more, about 10% or more, or about 15% or more) and/or about 20% or less (e.g., about 15% or less, about 10% or less, about 5% or less, about 2% or less, or about 1% or less) by weight, relative to the total weight of the composition. Thus in various embodiments, when the oil phase further includes an oil, the oil is present in an amount from about 0.5, 1, 2, 3, 4, 5, 6, 7, 8, 9, 10, 11, 12, 13, 14, 15, 16, 17, 18, 19 to about 20% by weight, including increments and intervals therein, relative to the total weight of the composition.

In some embodiments, when the oil phase further includes an oil, the oil can include natural oils. As used herein, the oil includes both oils and fats (which are generally solid at room temperature). Oils (including fats and butters) are glycerol esters composed of glycerol and fatty acids and are called triglycerides. The fatty acids can be saturated (no double bonds between the carbon atoms) or unsaturated (one or more double bonds), which can influence the stability and property of the oil. Oils having a high degree of saturated fatty acids (e.g., lauric, myristic, palmitic and stearic acids) include coconut oil, cottonseed oil, and palm oil. Oils with a high degree of unsaturated fatty acids (e.g., oleic, arachidonic, linoleic acid) include canola oil, olive oil, corn oil, almond oil, safflower oil, castor oil and avocado oil.

It is believed that saturated oils can be more stable and do not become rancid as quickly as unsaturated oils; while unsaturated oils can be smoother, more precious, less greasy, and better absorbed by the skin.

The oils in the oil phase can include natural butters such as shea butter, avocado butter, and/or cocoa butter. Natural butters can be good emollients and thickeners and, can have antioxidant and soothing properties (e.g., shea and avocado butters, which include phenolic compounds).

The oils can be of plant or animal origin. Examples of plant oils include linseed oil, camellia oil, macadamia nut oil, sunflower oil, apricot oil, soybean oil, arara oil, hazelnut oil, corn oil, olive oil, avocado oil, sasanqua oil, castor oil, safflower oil, jojoba oil, sunflower oil, almond oil, grapeseed oil, sesame oil, soybean oil, peanut oil, and mixtures thereof. Examples of animal oils include perhydrosqualene and squalane. In some embodiments, the oils can be botanical oils.

In some embodiments, the oil is olive oil, shea butter, linseed oil, camellia oil, macadamia nut oil, sunflower oil, apricot oil, soybean oil, arara oil, hazelnut oil, corn oil, avocado oil, sasanqua oil, castor oil, safflower oil, jojoba oil, sunflower oil, almond oil, grapeseed oil, sesame oil, soybean oil, peanut oil, avocado butter, cocoa butter, squalene, perhydrosqualene, or any combination thereof.

Waxes

In some embodiments, when the oil phase further includes a wax, the wax is present in an amount of about 1% to about 15% (e.g., about 1% to about 10%, about 5% to about 15%, about 5% to about 10%, about 10% to about 15%, or about 5% to about 8%) by weight, relative to the total weight of the composition. For example, the wax can be present in a given composition in an amount of about 1% or more (e.g., about 5% or more, about 8% or more, about 10% or more, or about 12% or more) and/or about 15% or less (e.g., about 12% or less, about 10% or less, about 8% or less, or about 5% or less) by weight, relative to the total weight of the composition. Thus in various embodiments, when the oil phase further includes a wax, the wax is present in an amount from about 1, 2, 3, 4, 5, 6, 7, 8, 9, 10, 11, 12, 13, 14 to about 15% by weight, including increments and intervals therein, relative to the total weight of the composition.

In some embodiments, when the oil phase further includes a wax, the wax includes natural waxes and synthetic waxes (which include emulsifying waxes). In some embodiments, waxes include esters of a fatty acid and a fatty alcohol, and can be complex mixtures of heavy hydrocarbons and fatty acids combined with esters. As used herein, waxes do not include glycerides. In general, waxes can be harder, less greasy and more brittle than fats, and are resistant to moisture, oxidization and microbial attack. Waxes can be characterized by a high melting point (50-100° C.). In some embodiments, waxes (e.g., beeswax) can be an emollient and thickener, and can provide emulsifying properties. In certain embodiments, waxes (e.g., carnauba and candelilla wax) can provide hardness and have relatively high melting points. Synthetic waxes can include hydroxyoctacosanyl hydroxystearate, cetyl palmitate (or cetyl ester wax).

Waxes can include animal waxes (e.g., beeswax, lanolin), plant waxes (e.g., carnauba wax, candililla wax, jojoba wax), mineral waxes (e.g., ozokerite, microcrystalline wax including mixed refined hydrocarbons, ceresin, petrolatum), and synthetic waxes.

In some embodiments, the wax is selected from synthetic wax, carnauba wax, microcrystalline waxes, ozokerites, hydrogenated jojoba oil, polyethylene waxes (e.g., “Performalene 400 Polyethylene” and “Performalene 500 Polyethylene,” available from New Phase Technologies), silicone waxes (e.g., poly(C₂₄-C₂₈)alkylmethyldimethylsiloxane, available as “Abil Wax 9810” sold by the company Goldschmidt), C₂₀-C₄₀ alkyl stearate (e.g., “Kester Wax K82H” available from the company Kester Keunen), stearyl benzoate, shellac wax, and mixtures thereof. For example, the oil phase can include a wax selected from carnauba wax, candelilla wax, ozokerites, hydrogenated jojoba oil and polyethylene waxes. In some embodiments, the wax is candelilla wax, ozokerite, or mixtures thereof. In some embodiments, the wax is a polyethylene wax, a synthetic wax, a carnauba wax, a microcrystalline wax, an ozokerite, a hydrogenated jojoba oil, or a combination thereof.

Esters

As discussed above, the compositions of the present disclosure can include linear fatty esters, or a combination of linear and branched fatty esters. In some embodiments, the at least one linear fatty ester is present in a given composition in an amount of about 1% to about 30% by weight (e.g., about 10% to about 25% by weight), relative to the total weight of the composition. For example, the at least one linear fatty ester can be present in an amount of about 1% or more (e.g., about 5% or more, about 10% or more, about 20% or more, or about 25% or more) and/or about 30% or less (e.g., about 25% or less, about 20% or less, about 10% or less, or about 5% or less) by weight in the composition, relative to the total weight of the composition. Thus, in various embodiments, the at least one linear fatty ester is present from about 1, 2, 3, 4, 5, 6, 7, 8, 9, 10, 11, 12, 13, 14, 15, 16, 17, 18, 19, 20, 21, 22, 23, 24, 25, 26, 27, 28, 29 to about 30% by weight, including increments and intervals therein, relative to the total weight of the composition.

In some embodiments, the at least one branched fatty ester is present in a given composition in an amount of about 1% to about 60% (e.g., about 20% to about 60%, about 25% to about 55%, about 1 to about 40%, or about 5% to about 35%) by weight, relative to the total weight of the composition. For example, the at least one branched fatty ester is present in a given composition in an amount of about 1% or more (e.g., about 5% or more, about 20% or more, about 25% or more, about 35% or more, or about 45% or more) and/or about 60% or less (e.g., about 45% or less, about 35% or less, about 25% or less, about 20% or less, or about 5% or less) by weight, relative to the total weight of the composition. As used herein, a “branched fatty ester” includes fatty esters having cyclic moieties (e.g., sugars) and/or branched ester (e.g., branched hydrocarbons). Thus, in various embodiments, the at least one branched fatty ester is present from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 to about 60% by weight, including increments and intervals therein, relative to the total weight of the composition.

In some embodiments, fatty esters are obtained by direct reaction of fatty acids with alcohols. Examples of the fatty esters of fatty alcohols and/or of fatty acids include fatty esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the fatty esters being greater than or equal to 10.

In some embodiments, the fatty esters can be monoesters, such as dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; cetyl palmitate, ethylhexyl palmitate, isocetyl stearate, octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

In some embodiments, fatty esters of C₄-C₂₂ dicarboxylic or tricarboxylic acids and of C₁-C₂₂ alcohols and esters of mono-, di- or tricarboxylic acids and of C₂-C₂₆ di-, tri-, tetra- and/or pentahydroxy alcohols can be used in the oil phase. For example, the esters can include diethyl sebacate; diisopropyl sebacate; diisopropyladipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityltetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and/or polyethylene glycol distearates.

In some embodiments, the fatty esters are selected from ethyl, isopropyl, myristyl, cetyl palmitate, stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; isocetyl stearate, dioctyl malate, hexyl laurate, 2-hexyldecyl laurate, isononyl isononanoate and cetyl octanoate. In some embodiments, the fatty esters are selected from cetyl palmitate, 2-ethylhexyl palmitate, isocetyl stearate, and any combination thereof. In some embodiments, the fatty esters are selected from cetyl palmitate, 2-ethylhexyl palmitate, and any combination thereof. In some embodiments, the fatty esters are selected from cetyl palmitate, isocetyl stearate, and any combination thereof. In some embodiments, the fatty ester is cetyl palmitate.

In some embodiments, the fatty esters can include sugar esters and diesters of C₆-C₃₀ fatty acids (e.g., C₁₂-C₂₂ fatty acids). As used herein, the term “sugar” refers to oxygen-bearing hydrocarbon-based compounds containing several alcohol functions, with or without aldehyde or ketone functions, and which contain at least 4 carbon atoms. These sugars may be monosaccharides, oligosaccharides or polysaccharides. Examples of suitable sugars include sucrose (or saccharose), glucose, galactose, ribose, fructose, maltose, mannose, arabinose, xylose and lactose, and derivatives thereof, alkyl derivatives thereof, such as methyl derivatives thereof, for instance methylglucose.

In some embodiments, the sugar esters of fatty acids are selected from the esters or mixtures of esters of sugars described previously and of linear or branched, saturated or unsaturated C₆-C₃₀ fatty acids (e.g., C₁₂-C₂₂ fatty acids). If the sugar esters of fatty acids are unsaturated, these compounds can include one to three conjugated or non-conjugated carbon-carbon double bonds. Esters according to this variant can include mono-, di-, tri-, tetraesters and polyesters, and mixtures thereof. These esters can be selected, for example, from oleates, laurates, palmitates, myristates, behenates, cocoates, stearates, linoleates, linolenates, caprates and arachidonates, or mixtures thereof such as oleo-palmitate, oleo-stearate and palmito-stearate mixed esters. In some embodiments, the sugar esters of fatty acids are monoesters and diesters, such as sucrose, glucose or methylglucose mono- or dioleates, stearates, behenates, oleopalmitates, linoleates, linolenates and oleo stearates.

In some embodiments, the fatty esters can include synthetic oils. As compared to the natural oils which have 3 chains of fatty acids (e.g., triglycerides), synthetic oils can have one fatty acid chain. Based on the variety of fatty acids and alcohols (e.g. butyl, isopropyl, ethylhexyl, myristyl, oleyl alcohol, cetyl), a wide range of synthetic oils can be obtained.

Other Fatty Materials

In some embodiments, in addition to the at least one linear fatty ester, the oil phase can further include any of a number of fatty materials (which can be an oil, an ester, or a wax; or components of an oil, an ester, or a wax), such as oils of animal or plant origin (described above), synthetic glycerides, esters of fatty alcohols and/or fatty acids (described above) other than animal or plant oils and synthetic glycerides, fatty alcohols, non-salified fatty acids, silicone oils and aliphatic hydrocarbons, and mixtures thereof. These fatty materials may be volatile or non-volatile. In some embodiments, the oil phase further includes a component selected from aliphatic hydrocarbons, plant oils, fatty alcohols, esters of fatty alcohols and/or fatty acids other than animal or plant oils and synthetic glycerides, and mixtures thereof. In certain embodiments, the oil phase further includes a component selected from aliphatic hydrocarbons.

Examples of aliphatic hydrocarbons include vaseline or petrolatum, naphthalenes, and the like; hydrogenated polyisobutene, isoeicosan, polydecenes, hydrogenated polyisobutenes such as Parleam, and decene/butene copolymer; and mixtures thereof. Examples of aliphatic hydrocarbons, can also include linear, branched, or cyclic C₆-C₁₆ lower alkanes. Examples of aliphatic hydrocarbons include hexane, undecane, dodecane, tridecane and isoparaffins such as isohexadecane and isodecane.

Examples of synthetic glycerides include caprylic/capric acid triglycerides, for instance those sold by the company Stearineries Dubois or those sold under the names Miglyol 810, 812 and 818 by the company Dynamit Nobel.

Examples of silicone oils include linear organopolysiloxanes such as dimethylpolysiloxane, methylphenylpolysiloxane, methylhydrogenpolysiloxane, and the like; cyclic organopolysiloxanes such as octamethylcyclotetrasiloxane, decamethylcyclopentasiloxane, dodecamethylcyclohexasiloxane, and the like; and mixtures thereof.

In some embodiments, the oil phase includes at least one fatty acid. The fatty acids can be in acidic form, can be saturated or unsaturated, can contain from 6 to 30 carbon atoms (e.g., from 9 to 30 carbon atoms), and can be optionally substituted (e.g., with one or more hydroxyl groups, such as 1 to 4 hydroxyl groups). The unsaturated fatty acids can include one to three conjugated or non-conjugated carbon-carbon double bonds. The fatty acid can include, for example, myristic acid, palmitic acid, stearic acid, behenic acid, oleic acid, linoleic acid, linolenic acid, and/or isostearic acid.

In some embodiments, the oil phase includes at least one fatty alcohol. As used herein, “fatty alcohol” refers to any saturated or unsaturated, linear or branched C₈-C₃₀ fatty alcohol, which can be optionally substituted (e.g., with one or more hydroxyl groups, such as 1 to 4 hydroxyl groups). In some embodiments, unsaturated fatty alcohols can include one to three conjugated or non-conjugated carbon-carbon double bonds. In some embodiments, the fatty alcohol is unsaturated and/or branched.

In some embodiments, the fatty alcohols are C₁₂-C₂₂ fatty alcohols. The fatty alcohols can include, for example, isostearyl alcohol, oleyl alcohol, linoleyl alcohol, undecylenyl alcohol, palmitoleyl alcohol, linolenyl alcohol, erucyl alcohol, and mixtures thereof. In some embodiments, cetyl alcohol, stearyl alcohol or a mixture thereof (e.g., cetearyl alcohol), as well as myristyl alcohol, can be used as a solid fatty material. In some embodiments, isostearyl alcohol can be used as a liquid fatty material. In one embodiment, the oil phase includes cetyl alcohol.

Non-Ionic Surfactants

As discussed above, the compositions of the present disclosure include at least one non-ionic surfactant. In some embodiments, the at least one non-ionic surfactant is present in a given composition in an amount of up to about 10% by weight of the undiluted non-ionic surfactant (e.g., up to about 8% by weight), relative to the total weight of the composition. For example, the at least one non-ionic surfactant can be present in the composition in an amount of about 1% or more (e.g., about 2% or more, about 3% or more, about 4% or more, about 5% or more, about 7% or more, or about 8% or more) and/or about 10% or less (e.g., about 8% or less, about 7% or less, about 5% or less, about 4% or less, about 3% or less, or about 2% or less) by weight of the undiluted non-ionic surfactant, relative to the total weight of the composition. In some embodiments, the at least one non-ionic surfactant is present in the composition in an amount of from about 4% to about 8% by weight of the undiluted non-ionic surfactant, relative to the total weight of the composition. Thus in various embodiments, when the at least one non-ionic surfactant is present in an amount from about 1, 2, 3, 4, 5, 6, 7, 8, 9 to about 10% by weight, including increments and intervals therein, relative to the total weight of the composition.

The nonionic surfactant of the present disclosure can have a hydrophilic-lipophilic balance (HLB) value of about 18.0 or less (e.g., from about 4.0 to about 18.0, from about 6.0 to about 15.0, from about 9.0 to about 13.0, or from about 9.0 to about 11.0). For example, the nonionic surfactant of the present disclosure can have a hydrophilic-lipophilic balance (HLB) value of about 18.0 or less (e.g., about 15.0 or less, about 13.0 or less, about 10.0 or less, about 5.0 or less, or about 3.0 or less). In some embodiments, the nonionic surfactant is selected from esters of polyols with fatty acids with a saturated or unsaturated chain containing for example from 8 to 24 carbon atoms (e.g., 12 to 22 carbon atoms), and alkoxylated derivatives thereof, such as glyceryl esters of a C₈-C₂₄ fatty acid or acids and alkoxylated derivatives thereof, polyethylene glycol esters of a C₈-C₂₄ fatty acid or acids and alkoxylated derivatives thereof, sorbitol esters of a C₈-C₂₄ fatty acid or acids and alkoxylated derivatives thereof, sugar (sucrose, glucose, alkylglycose) esters of a C₈-C₂₄ fatty acid or acids and alkoxylated derivatives thereof, ethers of fatty alcohols, ethers of sugar and a C₈-C₂₄ fatty alcohol or alcohols, and mixtures thereof.

In some embodiments, the non-ionic surfactant is selected from PEG-20 glyceryl triisostearate, PEG-7 glyceryl cocoate, PEG-20 methylglucoside sesquistearate, PG-5 dioleate, PG-4 diisostearate, PG-10 isostearate, PEG-8 stearate, PEG-8 isostearate, PEG-60 hydrogenated castor oil, and any combination thereof. In some embodiments, the non-ionic surfactant is PEG-20 glyceryl triisostearate.

Fillers

In some embodiments, the compositions of the present disclosure further include at least one filler. In some embodiments, the at least one filler is present in a given composition in an amount of up to about 20% by weight (e.g., up to about 15% by weight, up to about 10% by weight, up to about 5% by weight, or up to about 1% by weight), relative to the total weight of the composition. For example, the at least one filler can be present in the composition in an amount of about 0.1% or more (e.g., about 0.5% or more, about 1% or more, about 5% or more, about 10% or more, or about 15% or more) and/or about 20% or less (e.g., about 15% or less, about 10% or less, about 5% or less, about 1% or less, or about 0.5% or less) by weight, relative to the total weight of the composition. In some embodiments, the at least one filler is present in a given composition in an amount of from about 0.1% to about 10% (e.g., about 0.1% to about 1%, or about 0.5%) by weight, relative to the total weight of the composition. Thus in various embodiments, the at least one filler is present in an amount from about 1, 2, 3, 4, 5, 6, 7, 8, 9, 10, 11, 12, 13, 14, 15, 16, 17, 18, 19 to about 20% by weight, including increments and intervals therein, relative to the total weight of the composition.

Examples of fillers include silica silylate, kaolin, lauryl lysine, talc, bentone, cellulose beads (i.e., cellulobeads USF, available for example from Daito Kasei Kogyo), and any combination thereof. In some embodiments, the compositions include about 0.1% to about 1% (e.g., about 0.5%) by weight of silica silylate, relative to the total weight of the composition. In certain embodiments, the compositions include about 0.1% to about 1% (e.g., about 0.5%) by weight of silica silylate and about 0.1% to about 1% (e.g., about 0.5%) by weight of cellulose beads, relative to the total weight of the composition. Thus in various embodiments, the compositions are present in an amount from about 0.1, 0.2, 0.3, 0.4, 0.5, 0.6, 0.7, 0.8, 0.9 to about 1% by weight of silica silylate and/or cellulose beads, including increments and intervals therein, relative to the total weight of the composition.

The fillers (e.g., cellulose beads) can provide beneficial effects such as oil control, sebum control, sweat control, mattefying effects, and/or skin protective effects such as the ability to absorb pollutant particles to a composition.

Other Components

The compositions of the present disclosure can further include other components, such as phenylethyl resorcinol (i.e., Symwhite377, Symrise), capryloyl salicylic acid (i.e., LHA, Chimex), and a combination thereof. In some embodiments, the compositions of the present disclosure can include fragrances, antioxidants (e.g., vitamin E), plant extracts, and/or colorants.

In some embodiments, components such as fragrances, antioxidants, plant extracts, colorants, phenylethyl resorcinol, and capryloyl salicylic acid can each be present in a given composition in an amount of up to about 5% by weight (e.g., up to about 4% by weight, up to about 3% by weight, up to about 2% by weight, up to about 1% by weight, or up to about 0.5% by weight), relative to the total weight of the composition. For example, components such as fragrances, antioxidants, plant extracts, colorants, phenylethyl resorcinol, and capryloyl salicylic acid can each be present in a given composition in an amount of about 0.01% or more (e.g., about 0.05% or more, about 0.1% or more, about 0.5% or more, about 1% or more, about 1.5% or more, about 2% or more, about 3% or more, or about 4% or more) and/or about 5% or less (e.g., about 4% or less, about 3% or less, about 2% or less, about 1.5% or less, about 1% or less, about 0.5% or less, about 0.1% or less, or about 0.05% or less) by weight, relative to the total weight of the composition.

Formulations

In some embodiments, the composition includes about 5-10% by weight isocetyl stearate, about 45-55% by weight of ethylhexyl palmitate, about 0.5% by weight of silica silylate, about 15-25% by weight of cetyl palmitate, about 5-7% by weight of polyethylene, about 1% by weight of olive oil, about 1-2% by weight of shea butter, about 2% by weight of cetyl alcohol, about 0.1-2% by weight of caprylyl glycol, about 5% by weight of PEG-20 glyceryl triisostearate, about 5% by weight of neopentyl glycol diheptanoate, about 0.5% by weight of tocopherol, and about 0.1-1% by weight of plant extract and fragrance.

It is understood that for the compositions of the present disclosure, the sum of the percentages of the various components in any given composition is 100%, even though the sum of the ranges of the composition components may exceed 100%.

Properties of the Compositions

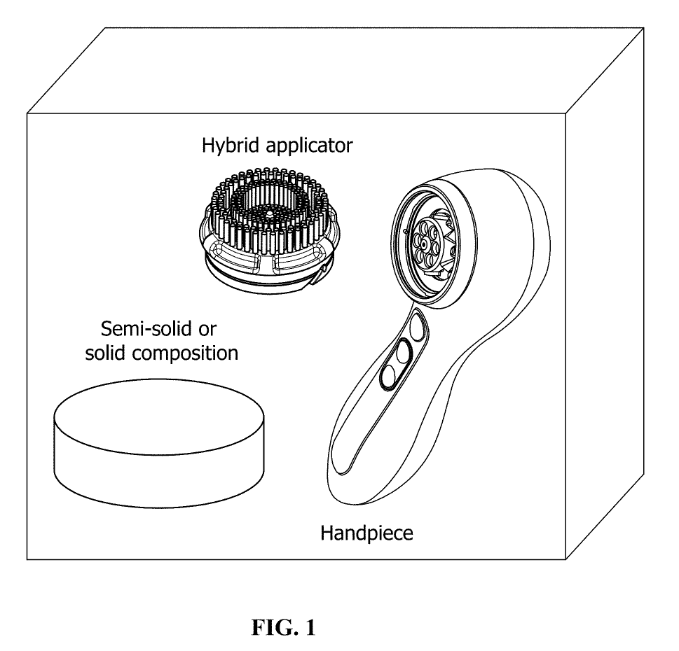

The compositions of the present disclosure can have a number of desirable properties. For example, the compositions can be molded via hot-pouring techniques. Examples of molded compositions disclosed herein are shown in FIG. 1. The compositions can retain a molded shape when subjected to a pop-out test (described below in Example 2) at ambient temperature. As used herein, “ambient temperature” refers to a temperature of from 20 to 30° C. As used herein, the term “about” indicates that the subject number can be increased or decreased by 5% and still fall within the embodiment described or claimed.

In some embodiments, the compositions can be solid at ambient temperature with a hardness of at least 35 g-force (e.g., at least 60 g-force). In some embodiments, the compositions have a hardness of 35 g-force or more (e.g., 40 g-force or more, 60 g-force or more, 80 g-force or more, or 100 g-force or more) and/or 130 g-force or less (e.g., 100 g-force or less, 80 g-force or less, 60 g-force or less, or 45 g-force or less), when the hardness is measured at ambient temperature. In certain embodiments, the compositions have a hardness of from 35 g-force to 130 g-force (e.g., from 60 g-force to 110 g-force), when the hardness is measured at ambient temperature. The hardness can be measured using a texture analyzer, using a procedure as described below in Example 1. In some embodiments, the hardness is measured after (e.g., 12 or more hours, or 12-24 hours after) forming a molded composition (e.g., via hot-pouring techniques) and can range from 60 g-force to 130 g-force, when measured at ambient temperature. In certain embodiments, the hardness is measured after performing a series of stability measurements at a variety of temperatures (e.g., 5° C., 10° C., 25° C., and 45° C.) on the molded composition; where the hardness can range from 35 g-force to 130 g-force, when measured at ambient temperature. As used herein, “ambient temperature” refers to a temperature of from 20 to 30° C.

In some embodiments, the compositions of the present disclosure can be used to remove makeup and/or dirt and can provide excellent cleansing ability. When cleansing with the compositions, the compositions can be used alone, manually, or with an implement or electronic device on wet or dry skin. In some embodiments, the compositions can be removed through emulsification with water or by wiping the compositions from the skin with a dry or wet substrate. When applied either manually or using an electronic device to a skin portion, the compositions can undergo a solid to cream (e.g., a creamy oil) transition over a period spanning from an initial application up to, but not limited to 5 minutes, at ambient temperature. As used herein, “cream” refers to a composition having a high viscosity that is softer than the solid and that is spreadable at ambient temperature.

The semi-solid or solid non-Newtonian composition according to the disclosure may be prepared according to techniques that are well known to those skilled in the art.

The semi-solid or solid non-Newtonian composition may be packaged in a pot, a tub, a tube or a unit dose package.

Unitary Package

Referring to FIG. 1, the cosmetic kit is packaged in a unitary package. The unitary package, for example, may include a bottle, tube, jar, case, box, sachet and carton.

Method

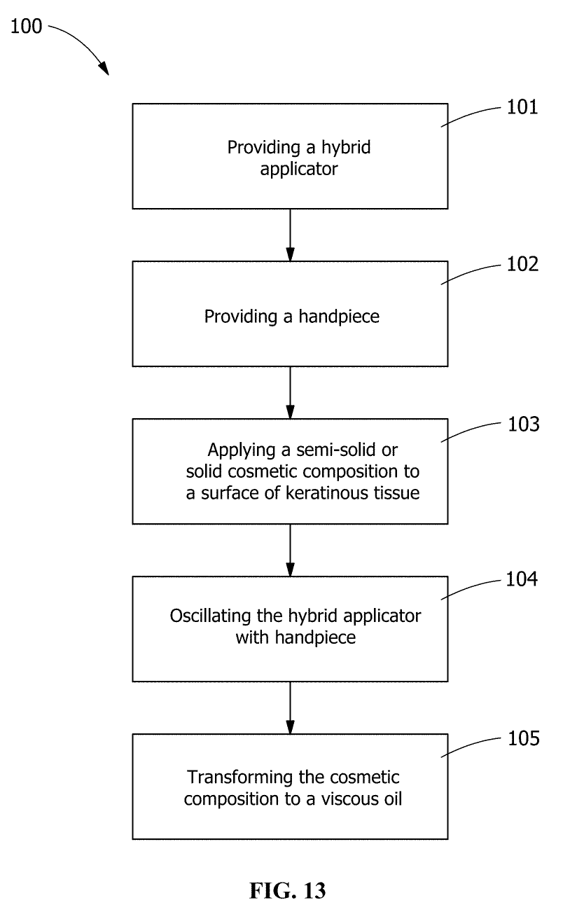

FIG. 13 shows a method 100, according to the present disclosure. Method 100 includes providing a hybrid applicator comprising a base and cleaning elements (step 101). The cleaning elements include bristled brush and a contact surface. The contact surface may be formed of elastomers including but not limited to TPE, TPU, silicone and combinations thereof. The method includes providing a handpiece comprising a body and a motor (step 102). The motor is arranged and disposed to provide oscillating motion to the hybrid applicator, when attached, relative to the body. The method further includes applying a semi-solid or solid non-Newtonian cosmetic composition (step 103). The cosmetic composition, for example, may include anhydrous based systems containing shear sensitive raw materials, aqueous based systems containing shear sensitive raw materials, blends containing shear sensitive raw materials or combinations thereof to a surface of keratinous tissue. The method further includes oscillating the hybrid applicator with the handpiece to transform the cosmetic composition to a viscous oil (steps 104 and 105). In some embodiments, as discussed above, the method may include three modes: initiation, transformation, and application modes. As discussed above, the transformation mode may further include two modes of operation: a cosmetic shear-thinning mode and a cosmetic melting mode.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

1. A cosmetic kit for applying a cosmetic composition to a surface of keratinous tissue, wherein the cosmetic kit is packaged in a unitary package, comprising:
a hybrid applicator comprising a base and cleaning elements comprising a bristled brush and a contact surface, the contact surface being formed of elastomers selected from the group consisting of TPE, TPU, silicone and combinations thereof;
a handpiece comprising a body and a motor, the body configured to receive the hybrid applicator, and the motor arranged and disposed to provide an oscillating motion to the hybrid applicator, when attached, relative to the body; and
a semi-solid or solid non-Newtonian cosmetic composition that is transformable to a spreadable consistency when subjected to oscillating contact with the hybrid applicator.

2. The cosmetic kit of claim 1, wherein the contact surface is formed of TPE/silicone.

3. The cosmetic kit of claim 1, wherein the bristled brush is selected from the group consisting of synthetic bristles, natural bristles, and combinations thereof.

4. The cosmetic kit of claim 1, wherein the motor does not deliver rotational and vibrational motions.

5. The cosmetic kit of claim 1, wherein the handpiece further includes an actuator selected from the group consisting of multimodal switch, knobs, and combinations thereof to provide three different modes: initiation, transformation, and application.

6. The cosmetic kit of claim 5, wherein the handpiece further includes a control switch to control the oscillating motion of the hybrid applicator at or above a frequency about 100 Hz with an amplitude in the range of 3-18° peak-to-peak in order to provide shear-thinning/ destructuring of the semi-solid or solid non-Newtonian cosmetic composition.

7. The cosmetic kit of claim 6, wherein control switch controls the oscillating motion of the hybrid applicator at or above a frequency about 175 Hz with an amplitude in the range of 3-18° peak-to-peak in order to provide melting of the semi-solid or solid non-Newtonian cosmetic composition.

8. A cosmetic kit for applying a cosmetic composition onto a surface of keratinous tissue, where the cosmetic kit is packaged in a unitary package, comprising:
a hybrid applicator comprising a base and cleaning elements comprising a bristled brush and a contact surface, the contact surface being formed of elastomers selected from the group consisting of TPE, TPU, silicone and combinations thereof;
a handpiece comprising a body and a motor, the body configured to receive the hybrid applicator, and the motor arranged and disposed to provide oscillating motion to the hybrid applicator, when attached, relative to the body; and
a semi-solid or solid non-Newtonian cosmetic composition selected from the group consisting of anhydrous based systems containing shear sensitive raw materials, aqueous based systems containing shear sensitive raw materials, blends containing shear sensitive raw materials, and combinations thereof, wherein the semi-solid or solid non-Newtonian cosmetic composition is transformable to a spreadable consistency when subjected to oscillating contact with the hybrid applicator.

9. The cosmetic kit of claim 8, wherein the spreadable consistency is in the form of a viscous oil.

10. The cosmetic kit of claim 8, wherein the bristled brush is selected from the group consisting of synthetic bristles, natural bristles, and combinations thereof.

11. The cosmetic kit of claim 8, wherein the motor does not deliver rotational and vibrational motions.

12. The cosmetic kit of claim 8, wherein the blends are selected from the group consisting of fatty esters, waxes, gums, ionic polymers and nonionic polymers.

13. The cosmetic kit of claim 8, wherein the handpiece further includes an actuator selected from the group consisting of multimodal switch, knobs, and combinations thereof to provide three different modes: initiation, transformation, and application.

14. The cosmetic kit of claim 13, wherein the handpiece further includes a control switch to control the oscillating motion of the hybrid applicator at or above a frequency about f₁ with an amplitude in the range of 3-18° peak-to-peak in order to provide shear-thinning/ destructuring of the semi-solid or solid non-Newtonian cosmetic composition.

15. The cosmetic kit of claim 14, wherein control switch controls the oscillating motion of the hybrid applicator at or above a frequency about f₂ with an amplitude in the range of 3-18° peak-to-peak in order to provide melting of the semi-solid or solid non-Newtonian cosmetic composition.

16. A method for cleansing and exfoliating a surface of keratinous tissue, comprising the steps of:
assembling the components of a cosmetic kit to provide a cosmetic applicator, the cosmetic kit comprising: a hybrid applicator, a handpiece, a cosmetic composition, the hybrid applicator comprising a base and cleaning elements comprising a bristled brush and a contact surface, the contact surface being formed of elastomers selected from the group consisting of TPE, TPU, silicone and combinations thereof, the handpiece comprising a body and a motor, the body configured to receive the hybrid applicator, and the motor arranged and disposed to provide oscillating motion to the hybrid applicator, when attached, relative to the body; the cosmetic composition comprising a semi-solid or solid non-Newtonian cosmetic composition selected from the group consisting of anhydrous based systems containing shear sensitive raw materials, aqueous based systems containing shear sensitive raw materials, blends containing shear sensitive raw materials and combinations thereof;
applying the cosmetic composition onto the hybrid applicator;
actuating the hybrid applicator by controlling a control switch and one of a multimodal switch and a knob to select one of two or more modes of operation, the modes of operation selected from at least (i) a cosmetic shear-thinning mode characterized by operation of the hybrid applicator at or above a frequency about 100 Hz with an oscillation amplitude in the range of 3-18° peak-to-peak, the cosmetic shear-thinning mode providing shear-thinning/destructing of the cosmetic composition to a consistency suitable for application, (ii) a cosmetic melting mode characterized by operation of the hybrid applicator at or above a frequency about 175 Hz with an oscillation amplitude in the range of 3-18° peak-to-peak, the cosmetic melting mode providing melting of the cosmetic composition to a consistency suitable for application; and (iii) an application mode characterized by operation alternating between two frequencies 100 Hz and 175 Hz with an oscillation amplitude in the range of 3-18° peak-to-peak, the application mode enabling application to the surface of keratinous tissue.

17. The method of claim 16, wherein the spreadable consistency is in the form of a viscous oil.

18. The method of claim 16, wherein the bristled brush is selected from the group consisting of synthetic bristles, natural bristles, and combinations thereof.

19. The method of claim 16, wherein the motor does not deliver rotational and vibrational motions.

20. The method of claim 16, wherein the blends are selected from the group consisting of fatty esters, waxes, gums, ionic polymers and nonionic polymers.
